(19) United States
(12) Patent Application Publication
(10) Pub. No.: US 2025/0386387 A1
(43) Pub. Date: Dec. 18, 2025

(54) PHYSIOLOGICAL MONITORING SYSTEM, PHYSIOLOGICAL MONITORING METHOD, AND COMPUTER READABLE MEDIUM

(71) Applicant: uCare Medical Electronics Co., Ltd., Miaoli County (TW)

(72) Inventors: Yi-Zhe Tsai, Miaoli County (TW); Fu-An Huang, Miaoli County (TW); Yuan-Pin Chang, Miaoli County (TW)

(73) Assignee: uCare Medical Electronics Co., Ltd., Miaoli County (TW)

(21) Appl. No.: 19/231,567

(22) Filed: Jun. 9, 2025

(30) Foreign Application Priority Data
Jun. 18, 2024 (TW) 113122471

(51) Int. Cl. H04W 76/14; A61B 5/00; H04B 1/3827; H04W 4/80

(52) U.S. Cl. CPC H04W 76/14; A61B 5/0004; A61B 5/002; A61B 5/0022; A61B 5/681; H04B 1/385; H04W 4/80

(57) ABSTRACT

A physiological monitoring system, a physiological monitoring method, and a computer readable medium are disclosed. The physiological monitoring method includes: obtaining physiological data by a wearable device through a sensor, wherein the wearable device is a Bluetooth central; advertising connection information by a terminal device, wherein the terminal device is a Bluetooth peripheral; receiving the connection information by the wearable device, and establishing a communication channel between the wearable device and the terminal device according to the connection information; transmitting the physiological data by the wearable device to the terminal device through the communication channel; and outputting a physiological status report by the terminal device according to the physiological data.

### CROSS-REFERENCE TO RELATED APPLICATION

This application claims the priority benefit of Taiwan application serial no. 113122471, filed on Jun. 18, 2024. The entirety of the above-mentioned patent application is hereby incorporated by reference herein and made a part of this specification.

### BACKGROUND

### Technical Field

This disclosure relates to a monitoring technology, and in particular to a physiological monitoring system, a physiological monitoring method, and a computer readable medium.

### Description of Related Art

Smartwatches have become a popular device for health monitoring. Smartwatches can monitor the user's physiological status in real time, which in turn provides the user with information related to the user's health.

Most of the smartwatches have the function to communicate with related devices (support the same operating system with the smartwatches) such as cell phones, and the smartwatch can provide the measured data to the cell phones for processing. But, different brands of smartwatches or cell phones may support (use) different operating systems (OS) or communication protocols. Thus, different brands of smartwatches and specific cell phones may not be compatible with each other. The smartwatch needs to relay the data to the cell phones via a relay device. However, the above relaying process increases the complexity of data transmission and transmission delay, resulting in a decrease in the reliability and efficiency of the monitoring system.

The information disclosed in this Background section is only for enhancement of understanding of the background of the described technology and therefore it may contain information that does not form the prior art that is already known to a person of ordinary skill in the art. Further, the information disclosed in the Background section does not mean that one or more problems to be resolved by one or more embodiments of the disclosure was acknowledged by a person of ordinary skill in the art.

### SUMMARY

In order to achieve one, part, or all of the above purposes or other purposes, a physiological monitoring system according to an embodiment of the disclosure includes a wearable device and a terminal device. The wearable device includes a sensor. The wearable device obtains physiological data through the sensor. The terminal device advertises connection information. The wearable device is a Bluetooth central, and the terminal device is a Bluetooth peripheral. The wearable device receives the connection information and establishes a communication channel between the wearable device and the terminal device according to the connection information. The wearable device transmits the physiological data to the terminal device through the communication channel. The terminal device outputs a physiological status report according to the physiological data.

In order to achieve one, part, or all of the above purposes or other purposes, a physiological monitoring method according to an embodiment of the disclosure includes the following. Physiological data is obtained by a wearable device through a sensor, where the wearable device is a Bluetooth central. Connection information is advertised by a terminal device, where the terminal device is a Bluetooth peripheral. The connection information is received by the wearable device, and a communication channel is established between the wearable device and the terminal device according to the connection information. The physiological data is transmitted by the wearable device to the terminal device through the communication channel. A physiological status report is outputted by the terminal device according to the physiological data.

In order to achieve one, part, or all of the above purposes or other purposes, a non-transitory computer readable medium according to an embodiment of the disclosure stores a transmitter program. A wearable device loads the transmitter program to perform: obtaining physiological data through a sensor, where the wearable device is a Bluetooth central; receiving connection information advertised by a terminal device, and establishing a communication channel between the wearable device and the terminal device according to the connection information, where the terminal device is a Bluetooth peripheral; and transmitting the physiological data to the terminal device through the communication channel to instruct the terminal device to output a physiological status report according to the physiological data.

In order to achieve one, part, or all of the above purposes or other purposes, a non-transitory computer readable medium according to an embodiment of the disclosure stores a receiver program. A terminal device loads the receiver program to perform: advertising connection information to a wearable device to instruct the wearable device to establish a communication channel between the wearable device and the terminal according to the connection information, where the wearable device is a Bluetooth central, and the terminal device is a Bluetooth peripheral; receiving physiological data obtained by the wearable device through a sensor from the wearable device; and outputting a physiological status report according to the physiological data.

Other objectives, features and advantages of the present invention will be further understood from the further technological features disclosed by the embodiments of the present invention wherein there are shown and described preferred embodiments of this invention, simply by way of illustration of modes best suited to carry out the invention.

### BRIEF DESCRIPTION OF THE DRAWINGS

The accompanying drawings are included to provide a further understanding of the disclosure, and are incorporated in and constitute a part of this specification. The drawings illustrate example embodiments of the disclosure and, together with the description, serve to explain the principles of the disclosure.

FIG. 1A, FIG. 1B, and FIG. 1C illustrate a schematic diagram of a physiological monitoring system according to an embodiment of the disclosure.

FIG. 2 is a schematic diagram of a Bluetooth protocol solution according to an embodiment of the disclosure.

FIG. 3 is a schematic diagram of a hypertext transfer protocol solution according to an embodiment of the disclosure.

FIG. 4 illustrates a flow chart of a physiological monitoring method according to an embodiment of the disclosure.

### DESCRIPTION OF THE EMBODIMENTS

It is to be understood that other embodiment may be utilized and structural changes may be made without departing from the scope of the present invention. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings.

The disclosure provides a physiological monitoring system, a physiological monitoring method, and a computer readable medium, capable of outputting physiological status report for user reference.

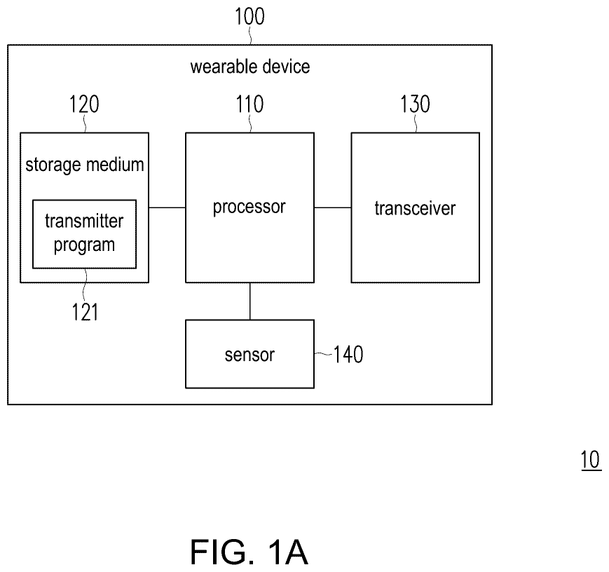

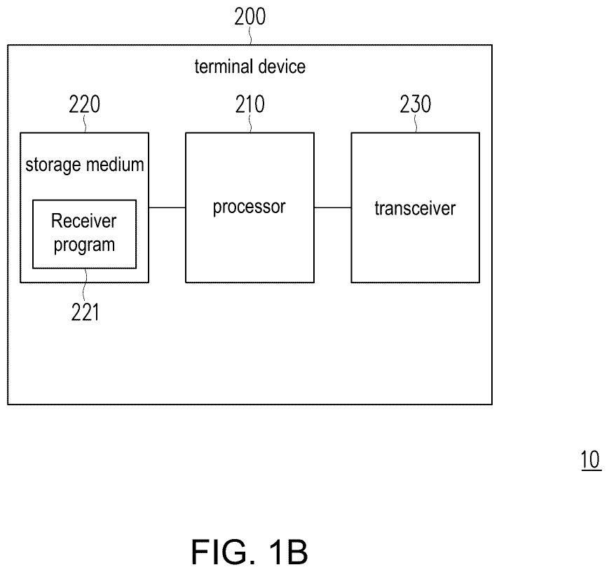

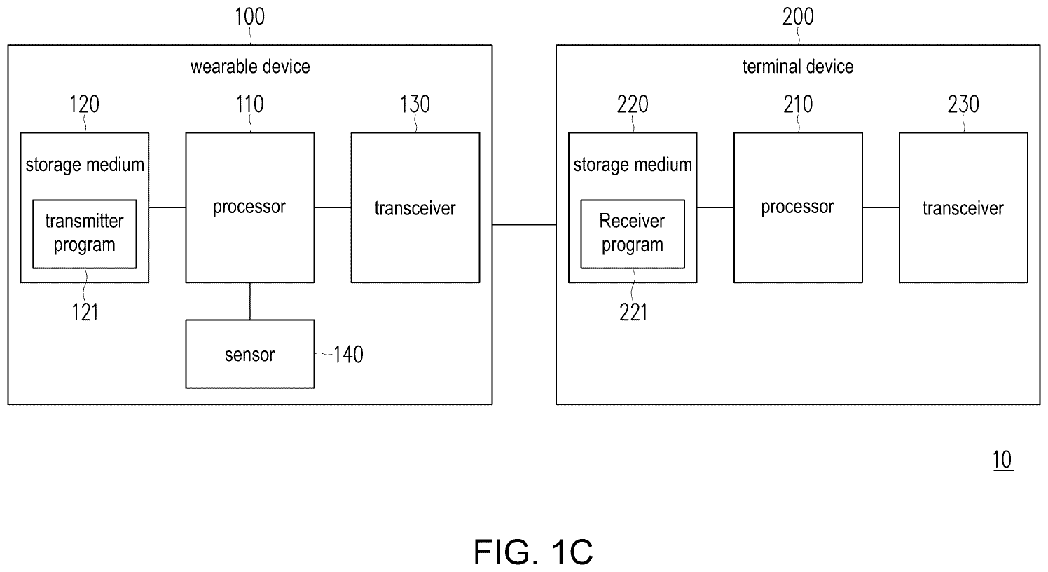

FIG. 1A, FIG. 1B, and FIG. 1C illustrate a schematic diagram of a physiological monitoring system according to an embodiment of the disclosure. In one embodiment, a physiological monitoring system 10 may include a wearable device 100, as shown in FIG. 1A. The wearable device 100 is, for example, a smartwatch. The wearable device 100 can realize the function of Bluetooth central through an application. As a Bluetooth central, the wearable device 100 can communicate and transmit data with a wider range of devices (i.e., Bluetooth peripheral), reducing the limitation of devices being of different operating systems or brands. In one embodiment, the physiological monitoring system 10 may include a terminal device 200, as shown in FIG. 1B. The terminal device 200 is, for example, a smartphone. In one embodiment, the physiological monitoring system 10 may include a wearable device 100 and a terminal device 200. Compared with the traditional monitoring system where the wearable device is Bluetooth peripheral and the terminal device is Bluetooth central, the wearable device 100 of the disclosure can realize the function of Bluetooth central through the application and the terminal device 200 can realize the function of Bluetooth peripheral through the application. In particular, Bluetooth peripheral is suitable for announcing its presence through an advertising function; Bluetooth central is suitable for finding nearby Bluetooth peripherals through a scan function.

The wearable device 100 may include a processor 110, a storage medium 120, a transceiver 130, and a sensor 140. The processor 110 is, for example, a central processing unit (CPU), or other programmable general-purpose or special-purpose micro control unit (MCU), microprocessor, or digital signal processor (DSP), programmable controller, application specific integrated circuit (ASIC), graphics processing unit (GPU), image signal processor (ISP), image processing unit (IPU), arithmetic logic unit (ALU), complex programmable logic device (CPLD), field programmable gate array (FPGA), or other similar components or a combination of the above components. The processor 110 may be coupled to the storage medium 120, the transceiver 130, and the sensor 140, and access and execute modules, programs, or applications stored in the storage medium 120.

The storage medium 120 may be a computer readable medium, such as a non-transitory computer readable medium. The storage medium 120 is, for example, any type of fixed or removable random access memory (RAM), read-only memory (ROM), or flash memory, hard disk drive (HDD), solid state drive (SSD), or similar components or a combination of the above components, used to store modules, programs, or applications that can be executed by the processor 110. In this embodiment, the storage medium 120 can store a transmitter program 121, the function of which is described later.

The transceiver 130 transmits or receives signals in a wireless or wired manner. The transceiver 130 may also perform, for example, low noise amplification, impedance matching, mixing, up or down frequency conversion, filtering, amplification, and similar operations. The processor 110 can be communicatively connected to the terminal device 200 through the transceiver 130. The transceiver 130 may support Bluetooth protocol or hypertext transfer protocol (HTTP), for example.

The sensor 140 may include, but is not limited to, a photoplethysmography (PPG) sensor or an accelerometer. When the wearable device 100 is worn on the user, the processor 110 can obtain physiological data of the user through the sensor 140. The physiological data of the user can include, but is not limited to, information such as heart rate or number of steps taken.

The terminal device 200 may include a processor 210, a storage medium 220, and a transceiver 230. The processor 210 is, for example, a CPU, or other programmable general-purpose or special-purpose MCU, microprocessor, DSP, programmable controller, ASIC, GPU, ISP, IPU, ALU, CPLD, FPGA, or similar components or a combination of the above components. The processor 210 may be coupled to the storage medium 220 and the transceiver 230, and access and execute modules, programs, or applications stored in the storage medium 220.

The storage medium 220 may be a computer readable medium, such as a non-transitory computer readable medium. The storage medium 220 is, for example, any type of fixed or removable random access memory, read-only memory, flash memory, hard drive, solid state drive, or similar components or a combination of the above components, used to store modules, programs, or applications that can be executed by the processor 210. In this embodiment, the storage medium 220 can store a receiver program 221, the function of which is described later.

The transceiver 230 transmits or receives signals in a wireless or wired manner. The transceiver 230 may also perform, for example, low noise amplification, impedance matching, mixing, up or down frequency conversion, filtering, amplification, and similar operations. The processor 210 can be communicatively connected to the wearable device 100 through the transceiver 230. The transceiver 230 may support Bluetooth protocol or HTTP, for example.

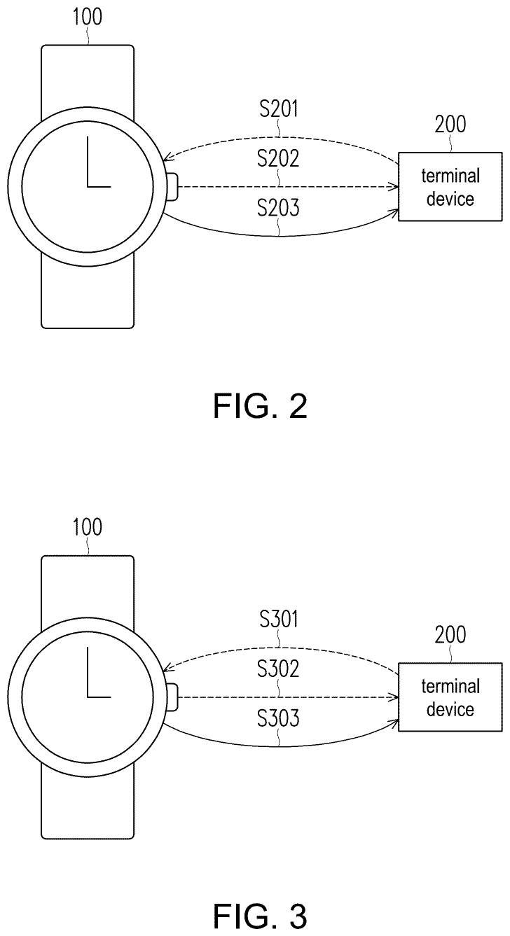

In the disclosure, the wearable device 100 and the terminal device 200 may use the Bluetooth protocol solution and/or the HTTP solution for communication and data transmission. FIG. 2 is a schematic diagram of a Bluetooth protocol solution according to an embodiment of the disclosure. Please refer to FIG. 1A to FIG. 1C and FIG. 2 at the same time. In step S201, the terminal device 200 can serve as a Bluetooth peripheral and the receiver program 221 advertises connection information through the transceiver 230 to notify nearby Bluetooth devices. The connection information may contain service data of the Bluetooth device (e.g., Bluetooth protocol). The wearable device 100 can serve as a Bluetooth central and the transmitter program 121 can receive the connection information through the transceiver 130 and obtain the service data from the connection information.

In step S202, the transmitter program 121 of the wearable device 100 can establish a communication channel between the wearable device 100 and the terminal device 200 according to the connection information. Specifically, if the transmitter program 121 detects that the connection information includes the service data of the Bluetooth protocol, the transmitter program 121 can establish a communication channel according to the service data.

In step S203, the transmitter program 121 of the wearable device 100 may transmit the physiological data obtained by the sensor 140 to the terminal device 200 through the communication channel. The receiver program 221 of the terminal device 200 can generate a physiological status report according to the physiological data, and output the physiological status report for user reference. The physiological status report can contain information related to the heart rate or number of steps taken of the user.

In one embodiment, the transmitter program 121 can execute a feature write command of the Bluetooth protocol to write the physiological data to the terminal device 200 (e.g., to the storage medium 220 of the terminal device 200) in the form of feature write. In this way, the physiological monitoring system 10 can write the physiological data (obtained by the sensor 140 of the wearable device 100) to the terminal device 200 without the terminal device 200 executing a notify command or a read command. That is, the wearable device 100 can serve as a Bluetooth central instead of a Bluetooth peripheral to write the physiological data to the terminal device 200, and the terminal device 200 can serve as a Bluetooth peripheral instead of a Bluetooth central to obtain the physiological data from the wearable device 100. Compared with a traditional wearable device as a Bluetooth peripheral that can only transmit data to a specific Bluetooth central (where the physiological data obtained by the wearable device is read by a terminal device), the wearable device 100, as a Bluetooth central, can communicate with a wider range of devices (i.e., peripheral) for communication and data transmission. For example, even if the wearable device 100 and the terminal device 200 respectively support (installed with) different operating systems, the wearable device 100 can still transmit the obtained physiological data to the terminal device 200 through the Bluetooth protocol.

FIG. 3 is a schematic diagram of a hypertext transfer protocol solution according to an embodiment of the disclosure. In step S301, the terminal device 200 can serve as a Bluetooth peripheral and the receiver program 221 advertises the connection information through the transceiver 230. The connection information may include an Internet protocol (IP) address. The wearable device 100 can serve as a Bluetooth central and the transmitter program 121 can receive the connection information through the transceiver 130 and obtain an IP address from the connection information.

In step S302, the transmitter program 121 of the wearable device 100 can establish a communication channel between the wearable device 100 and the terminal device 200 according to the connection information. Specifically, the transmitter program 121 can send instructions to the terminal device 200 according to the IP address to establish a communication channel (network communication channel) between the wearable device 100 and the terminal device 200.

In step S303, the transmitter program 121 of the wearable device 100 may transmit the physiological data obtained by the sensor 140 to the terminal device 200 through the communication channel. The receiver program 221 of the terminal device 200 can generate a physiological status report according to the physiological data, and output the physiological status report for user reference. The physiological status report can contain information related to the heart rate or number of steps taken of the user.

In one embodiment, the transmitter program 121 of the wearable device 100 can execute a post command of an HTTP to transmit the physiological data to the terminal device 200. In one embodiment, the wearable device 100 can serve as a network client that supports HTTP, and the terminal device 200 can serve as a network server that supports HTTP. Compared with a traditional wearable device as a Bluetooth peripheral that can only transmit data to a specific Bluetooth central, the wearable device 100 can communicate and transmit data with a variety of devices (such as network servers) through the Internet. For example, even if the wearable device 100 and the terminal device 200 respectively support different operating systems, the wearable device 100 can still transmit the obtained physiological data to the terminal device 200 through HTTP.

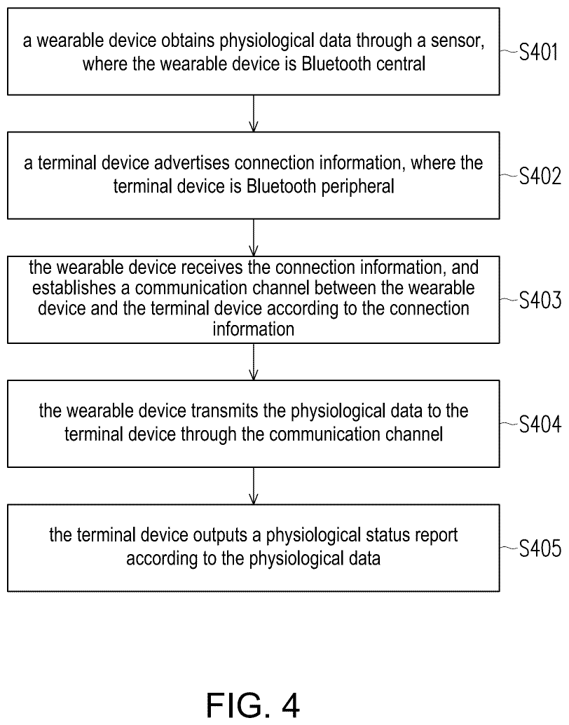

FIG. 4 illustrates a flow chart of a physiological monitoring method according to an embodiment of the disclosure, where the physiological monitoring method can be implemented by the physiological monitoring system 10 shown in FIG. 1A, FIG. 1B, or FIG. 1C. In step S401, the physiological data is obtained by the wearable device through the sensor, where the wearable device is Bluetooth central. In step S402, the terminal device advertises the connection information, where the terminal device is Bluetooth peripheral. In step S403, the wearable device receives the connection information, and establishes a communication channel between the wearable device and the terminal device according to the connection information. In step S404, the wearable device transmits the physiological data to the terminal device through the communication channel. In step S405, the terminal device outputs a physiological status report according to the physiological data.

In summary, the wearable device of the physiological monitoring system of the disclosure can establish a communication channel through the connection information advertised by the terminal device, and transmit the physiological data measured by the wearable device to the terminal device through the communication channel. The terminal device can generate a physiological status report according to the physiological data for user reference. The physiological monitoring system of the embodiment of the disclosure has at least one of the following advantages: compared with the traditional wearable device as a Bluetooth peripheral, the physiological monitoring system of the disclosure configures the wearable device as a Bluetooth central, so that the wearable device can interact with more diverse peripheral devices for communication or data transmission. In other words, the wearable device of the disclosure can communicate with a wider variety of terminal devices (that is, terminal devices as Bluetooth peripherals). In addition, the wearable device of the disclosure can directly transmit data to the terminal device without going through a relay device. Thus, compared with the traditional solution of a wearable device forwarding data to a terminal device through a relay device, the physiological monitoring system of the disclosure has higher reliability, lower transmission delay, and higher efficiency.

The foregoing description of the preferred embodiments of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to best explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like does not necessarily limit the claim scope to a specific embodiment, and the reference to particularly preferred exemplary embodiments of the invention does not imply a limitation on the invention, and no such limitation is to be inferred. The invention is limited only by the spirit and scope of the appended claims. Moreover, these claims may refer to use “first”, “second”, etc. following with noun or element. Such terms should be understood as a nomenclature and should not be construed as giving the limitation on the number of the elements modified by such nomenclature unless specific number has been given. The abstract of the disclosure is provided to comply with the rules requiring an abstract, which will allow a searcher to quickly ascertain the subject matter of the technical disclosure of any patent issued from this disclosure. It is submitted with the understanding that it will not be used to interpret or limit the scope or meaning of the claims. Any advantages and benefits described may not apply to all embodiments of the invention. It should be appreciated that variations may be made in the embodiments described by persons skilled in the art without departing from the scope of the present invention as defined by the following claims. Moreover, no element and component in the present disclosure is intended to be dedicated to the public regardless of whether the element or component is explicitly recited in the following claims.

What is claimed is:

1. A physiological monitoring system, comprising:
a wearable device, comprising a sensor, wherein the wearable device obtains physiological data through the sensor; and
a terminal device, advertising connection information, wherein the wearable device is a Bluetooth central, and the terminal device is a Bluetooth peripheral, wherein
the wearable device receives the connection information, and establishes a communication channel between the wearable device and the terminal device according to the connection information, wherein
the wearable device transmits the physiological data to the terminal device through the communication channel, and
the terminal device outputs a physiological status report according to the physiological data.

2. The physiological monitoring system according to claim 1, wherein the connection information comprises service data, wherein the wearable device establishes the communication channel in response to detecting the service data.

3. The physiological monitoring system according to claim 2, wherein the wearable device executes a feature write command of a Bluetooth protocol to transmit the physiological data to the terminal device.

4. The physiological monitoring system according to claim 1, wherein the connection information comprises an Internet Protocol address, wherein the wearable device establishes the communication channel according to the Internet Protocol address.

5. The physiological monitoring system according to claim 4, wherein the terminal device advertises the Internet Protocol address through a Bluetooth protocol.

6. The physiological monitoring system according to claim 4, wherein the wearable device executes a post command of a hypertext transfer protocol to transmit the physiological data to the terminal device.

7. The physiological monitoring system according to claim 4, wherein the wearable device is a network client, and the terminal device is a network server.

8. The physiological monitoring system according to claim 1, wherein the sensor comprises a photoplethysmography sensor or an accelerometer.

9. A physiological monitoring method, comprising:
obtaining physiological data by a wearable device through a sensor, wherein the wearable device is a Bluetooth central;
advertising connection information by a terminal device, wherein the terminal device is a Bluetooth peripheral;
receiving the connection information by the wearable device, and establishing a communication channel between the wearable device and the terminal device according to the connection information;
transmitting the physiological data by the wearable device to the terminal device through the communication channel; and
outputting a physiological status report by the terminal device according to the physiological data.

10. A non-transitory computer readable medium, storing a transmitter program, wherein a wearable device loads the transmitter program to perform:
obtaining physiological data through a sensor, wherein the wearable device is a Bluetooth central;
receiving connection information advertised by a terminal device, and establishing a communication channel between the wearable device and the terminal device according to the connection information, wherein the terminal device is a Bluetooth peripheral; and
transmitting the physiological data to the terminal device through the communication channel to instruct the terminal device to output a physiological status report according to the physiological data.

11. A non-transitory computer readable medium, storing a receiver program, wherein a terminal device loads the receiver program to perform:
advertising connection information to a wearable device to instruct the wearable device to establish a communication channel between the wearable device and the terminal according to the connection information, wherein the wearable device is a Bluetooth central, and the terminal device is a Bluetooth peripheral;
receiving physiological data obtained by the wearable device through a sensor from the wearable device; and
outputting a physiological status report according to the physiological data.
